(19) United States
(12) Patent Application Publication
(10) Pub. No.: US 2008/0242912 A1
(43) Pub. Date: Oct. 2, 2008

(54) Methods and Apparatus for Providing a High Purity Acetylene Product

(76) Inventors: Olivier Letessier, San Jose, CA (US); Richard J. Udischas, Newark, DE (US); James J.F. McAndrew, Glen Mills, PA (US); Regis Zils, Thionville (FR); Fabrice Delcorso, Saclay (FR); Rajat Agrawal, Newark, DE (US)

Correspondence Address: AIR LIQUIDE;Intellectual Property, 2700 POST OAK BOULEVARD, SUITE 1800, HOUSTON, TX, 77056, US

(21) Appl. No.: 11/942,361

(22) Filed: Nov. 19, 2007

Related U.S. Application Data
(60) Provisional application No. 60/908,908, filed on Mar. 29, 2007.

(51) Int. Cl. C07C 7/12; B01D 35/30; B01D 35/18

(52) U.S. Cl. 585/821; 585/820; 585/822; 210/257.1; 210/137; 210/203; 210/177

(57) ABSTRACT

Methods and apparatus for the purification and distribution of acetylene. Acetylene is removed from an acetylene storage device, and provided to a purifier where solvent is removed. The purified acetylene is then provided to a semiconductor processing tool.

### CROSS REFERENCE TO RELATED APPLICATIONS

The present application claims the benefit of U.S. Provisional Application Ser. No. 60/908,908, filed Mar. 29, 2007, herein incorporated by reference in its entirety for all purposes.

### BACKGROUND

1. Field of the Invention

This invention relates generally to the field of semiconductor fabrication. More specifically, the invention relates to methods and apparatus for providing high purity acetylene to semiconductor processing tools.

2. Background of the Invention

Acetylene is a hydrocarbon useful in many industrial applications. Typically acetylene is dissolved in a solvent and stored in conventional cylinders which are filled with a porous media. This is done to prevent the explosive decomposition of acetylene. Because acetylene is thermodynamically unstable as compared to is constituent elements (carbon and hydrogen) decomposition, once initiated, can lead to an explosion. Dissolving acetylene in acetone and using porous storage media greatly reduces this hazard, primarily by providing thermal mass and reducing the free volume in the cylinder.

When acetylene stored this way is withdrawn from a cylinder, a small amount of solvent may be entrained in the withdrawn acetylene. The amount of entrained solvent is dependent upon factors such as the cylinder pressure, temperature and the rate at which the acetylene is withdrawn from the cylinder. The amount of entrained solvent may also change as the total amount of acetylene stored in the cylinder decreases. It is possible that the amount of solvent in the acetylene can range from about 0.1% to about 1%, or even depending on the flow rate of withdraw, up to about 10%.

The presence of solvent in acetylene may be quite detrimental to some processes used in the chemical and semiconductor industries (including processes used for producing logic components, memory components, flat panel components and photovoltaic components) which require a high purity acetylene supply. Many of these processes occur at very high temperatures, and at these temperatures solvents will often result in the formation of oxygen, which can be highly undesirable from a process standpoint.

Methods exist for supplying acetylene without solvent to industrial applications. For instance, acetylene packaged without solvent is available, but it is only stored in gaseous state (at pressures below about 35 psig), making the amount of volume capable to be provided very low. Likewise, it is possible to produce acetylene on site in order to avoid storing the acetylene in a solvent. However, producing acetylene on site through hydrocarbon cracking is a capital intensive operation and only usually practical for very high consumption rates. Another method of on site production of acetylene is through the hydrolysis of calcium carbide, however, this method results in acetylene that may be contaminated with water vapor and with any contaminants present in the water used for the hydrolysis.

Consequently, there exists a need for improved methods and apparatus for providing a high purity acetylene product to a semiconductor processing tool by removing solvent from acetylene withdrawn from a storage source.

### BRIEF SUMMARY

The invention provides novel methods and apparatus for producing a high purity acetylene product by removing a solvent from an acetylene stream. The disclosed methods and apparatus produce a purified acetylene product by removing solvent from an acetylene stream with an adsorption type purifier.

In an embodiment, a method of providing a high purity acetylene product to a semiconductor processing tool comprises providing an acetylene source which contains acetylene dissolved in a solvent. A stream comprising acetylene and the solvent is withdrawn from the acetylene source and the solvent is then removed from the withdrawn stream by purifying with an adsorption type purifier, thereby producing a purified acetylene stream. This purified acetylene stream is then provided to at least one semiconductor processing tool.

In another embodiment, an apparatus for producing a high purity acetylene product by removing solvent from a stream of acetylene comprises an acetylene storage source, wherein the storage source contains acetylene which is dissolved in a solvent. An acetylene distribution line is also provided, where the distribution line connects the acetylene storage source with a semiconductor processing tool. A first acetylene purifier is provided on the distribution line, where the first purifier comprises an adsorption medium suitable to remove at least part of the solvent from the acetylene, and where the first purifier receives a stream of acetylene from the storage source, and where it produces a purified stream of acetylene to be sent to the semiconductor processing tool.

Other embodiments of the current invention may include, without limitation, one or more of the following features:
a pressure reducing device is provided on the distribution line, located between the storage source and the first purifier;
the pressure reducing device reduces the pressure to less than about 50 psig, preferably to less than about 20 psig;
the withdrawn acetylene stream has a residence time in the first adsorption type purifier between about 30 and about 600 seconds;
the purified acetylene stream is monitored to determine the amount of solvent present;
a second adsorption type purifier is provided in parallel to the first adsorption type purifier, the flow of the reduced pressure stream is switched from the first purifier to the second purifier, and the first purifier is regenerated;
the flow is switched selectively between the first and second purifiers when the detector detects an increase in the amount of solvent present in the purified stream of acetylene;
the flow is switched selectively between the first and second purifiers based upon the amount of purified acetylene provided to the semiconductor processing tool;
the first purifier is regenerated by passing an inert gas through the purifier to strip collected solvent from the adsorbent;
the first purifier is regenerated by heating the purifier bed, and/or the inert gas to a temperature between about 50 C and about 200 C;
the purified acetylene stream is provided to at least one semiconductor processing tool from the second purifier while the first purifier is regenerating;
the first and second purifiers are adsorption type purifiers comprising an activated carbon-based adsorbent, and the adsorbent is substantially free of metallic content;
the activated carbon based adsorbent is substantially free of metallic content, for instance, it contains less than about 100 ppm of iron;
the purified acetylene stream is substantially particle free, containing less than about 1 particle per milliliter which is greater than about 0.3 micron in size;
the solvent comprises either acetone or dimethyl-formamide (DMF);
the stream from the acetylene source is withdrawn at a rate between about 0.5 lpm and about 20 lpm;
the stream of purified acetylene contains less than about 50 ppm of solvent;
the pressure of the withdrawn stream is reduced to less than about 20 psig;
additional impurities are removed from the withdrawn acetylene stream with the first purifier, such that the purified acetylene stream comprises less than about 1 ppm of phosphine and sulfur compounds;
a pressure reducing device is located on the distribution line between the storage source and the first purifier;
a detector is located on the distribution line after the first purifier, wherein the detector is suitable to the purified acetylene stream;
a second purifier located on the distribution line in parallel to the first purifier;
a diverter valve, suitable to divert the flow of acetylene from the storage source to either the first or second purifier;
a source of inert gas and an inert gas line which connects the source of inert gas to the first and second purifiers so as to allow inert gas to selectively flow through a purifier when the flow of acetylene is diverted from that purifier;
a filter, suitable to remove particles from the purified stream of acetylene, located downstream of the first and second purifiers;
a flow controller on the distribution line after the first purifier and before the semiconductor processing tool; and
a heating source which is suitable to heat the inert gas, the first purifier, or the second purifier to a temperature between about 50 C and about 200 C.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 illustrates a schematic representation of one embodiment according to the current invention;

FIG. 2 illustrates a graphical representation of a cylinder depletion test for an typical cylinder containing acetylene dissolved in acetone; and

FIG. 3 illustrates FTIR spectra comparing typical acetylene and acetylene as purified according to one embodiment of the current invention.

### DESCRIPTION OF PREFERRED EMBODIMENTS

Generally, the current invention relates to a method of providing a high purity acetylene product to a semiconductor processing tool comprises providing an acetylene source which contains acetylene dissolved in a solvent. A stream comprising acetylene and the solvent is withdrawn from the acetylene source the solvent is then removed from the withdrawn stream by purifying with an adsorption type purifier, thereby producing a purified acetylene stream. This purified acetylene stream is then provided to at least one semiconductor processing tool. The current invention also generally relates to an apparatus for producing a high purity acetylene product by removing solvent from a stream of acetylene, the apparatus comprising an acetylene storage source, wherein the storage source contains acetylene which is dissolved in a solvent. An acetylene distribution line is also provided, where the distribution line connects the acetylene storage source with a semiconductor processing tool. A first acetylene purifier is on provided on the distribution line, where the first purifier comprises an adsorption medium suitable to remove at least part of the solvent from the acetylene, and where the first purifier receives a stream of acetylene from the storage source and it produces a purified stream of acetylene to be sent to the semiconductor processing tool.

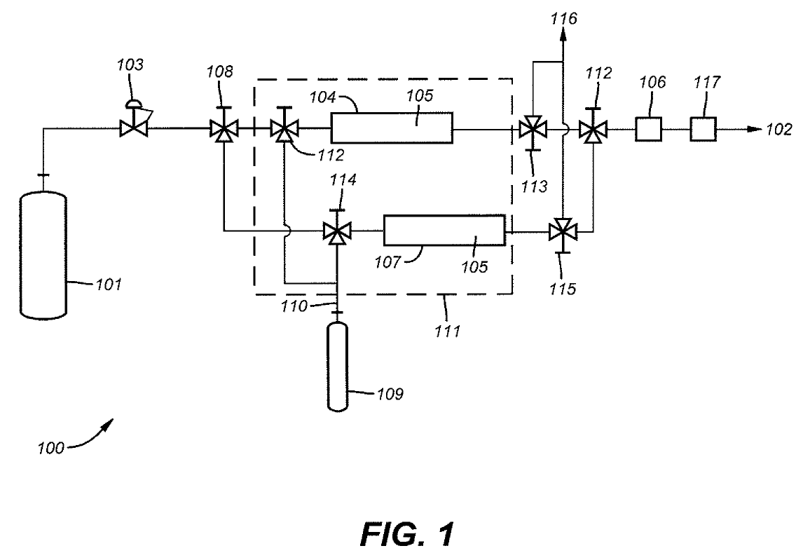

Referring now to FIG. 1, embodiments of the method and apparatus according to the current invention are described hereafter. An acetylene purification and distribution system 100 is shown. Acetylene storage source 101 contains acetylene dissolved in a solvent. In some embodiments, acetylene storage source 101 may be a conventional storage source such as a cylinder or a bank of cylinders. The storage source 101 may be filled with a porous media which contains a solvent such as acetone or dimethylformamide (DMF) in which the acetylene is dissolved. Acetylene purification and distribution system 100 is situated so as to distribute acetylene from the storage source 101, to a semiconductor processing tool 102. An acetylene stream, which may also contain some of the solvent, is withdrawn from the storage source 101, and provided to the distribution system 100. While in some embodiments, the acetylene stream may be withdrawn from the storage source 101 at a flow rate between about 0.5 liters per minute (lpm) and about 20.0 lpm, it is possible that the acetylene may be withdrawn from the storage source 101 at flow rate of about 100 lpm.

Located downstream from storage source 101 is pressure reducing device 103, which reduces the pressure of the stream of acetylene to less than about 50 psig, and preferably to less than about 20 psig. In some embodiments pressure reducing device 103 may be a conventional pressure reducing device such as a regulator valve.

A first purifier 104 is located down stream of the pressure reducing device 103. First purifier 104 contains an adsorption medium 105, and is suitable to remove at least part of the solvent contained in the acetylene stream, thereby purifying the stream to create a purified acetylene stream suitable to be provided the semiconductor processing tool 102. In some embodiments, first purifier 104 may be sized so as to provide a residence time, for the acetylene passing through it, of about 30 seconds and about 600 seconds.

In some embodiments, the adsorption medium 105 may be an activated carbon type adsorbent (e.g. activated carbon from coconut shell), which is substantially free from metallic content. Preferably, the adsorbent will contain less than about 100 ppm of iron. One method for removing the metallic content from the adsorbent is by washing the adsorbent with acid.

In some embodiments, the purified acetylene stream from the first purifier 104 comprises low levels of impurities. For instance, the purified acetylene stream may contain less than about 100 ppm, preferably less than about 50 ppm, and even more preferably less than about 10 ppm of the solvent. In addition to solvent, the first purifier 104 may remove additional impurities from the withdrawn acetylene, for instance, phosphine and sulfur compounds (e.g. hydrogen sulfide) may be removed. In some embodiments, the purified acetylene stream may contain less than about 10 ppm, and preferably less than about 1 ppm of either phosphine or sulfur compounds.

In some embodiments, the purified acetylene stream is substantially particle free, comprising less than about 1 particle per milliliter which is greater than 0.3 microns in size. A filter 118, suitable to remove particles from the purified acetylene stream, may be located downstream of the first purifier 104.

In some embodiments, detector 106 is located downstream of first purifier 104. Detector 106 may monitor the amount of solvent present in the purified acetylene stream before the purified acetylene stream is sent to the semiconductor processing tool 102. This allows confirmation that the solvent has been sufficiently removed from purified acetylene stream, and also gives a measure of the effectiveness of the first purifier 104 for removing solvent. Detector 106 may also monitor the amount of acetylene purified by the system 100, by either measuring flow rate or throughput of the purified acetylene stream being sent to semiconductor processing tool 102. In some embodiments, detector 106 may be a conventional type detector such as a Fourier Transform Infrared Spectroscopy (“FTIR”) type detector, or a flow rate meter. Detector 106 may be located directly on the main flow path for distribution system 100, or detector 106 may be situated off the main flow path such that some part of the purified acetylene stream is diverted to detector 106.

In some embodiments, a second purifier 107, which may be similar or identical in construction to first purifier 104 as described above, is located in parallel to the first purifier 104. By locating first purifier 104 and second purifier 107 in parallel, the flow of acetylene from the acetylene storage source 101 may be diverted between the first purifier 104 and second purifier 107. In some embodiments, diverting the flow between the purifiers is accomplished by providing a three-way type diverter valve 108 downstream of the pressure reducing valve 103, and upstream of both first purifier 104 and second purifier 107. In some embodiments, the flow of acetylene from the acetylene source 101 is selectively switched between the first purifier 104 and the second purifier 107 when the detector 106 detects an increase in the amount of solvent present in the purified acetylene stream. An increase in the amount of solvent may be indicative of the fact that the first purifier 104 needs to be regenerated. In some embodiments, the flow of acetylene may be selectively switched based upon other criteria, including without limitation, the amount of purified acetylene provided to semiconductor tool 102, the flow rate of the purified acetylene, or the time since the last regeneration of first purifier 104.

In some embodiments, a flow controller 117 is provided. Flow controller 117 is suitable to control the flow rate of the purified acetylene stream to the semiconductor processing tool 102, and may be a conventional flow controller such as a mass flow controller (“MFC”). Flow controller 117 may be located downstream from first purifier 104, and upstream of semiconductor processing tool 102.

In some embodiments an inert gas source 109 and an inert gas line 110 may also be provided. Inert gas source 109 may be a conventional source of inert gas, such as a cylinder, or may be a connection to another existing inert gas distribution system. Inert gas line 110 serves to supply inert gas (e.g. argon, nitrogen, helium, or carbon dioxide) to first purifier 104 and second purifier 107.

In some embodiments, the first purifier 104 is regenerated. This may be done because detector 106 detects an increase in solvent, or it may be done routinely based upon system parameters such as total operating time or total distribution system 100 throughput.

One method to regenerate the first purifier 104 is heating by the first purifier 104, and passing the inert gas from inert gas source 109 through the first purifier 104. Flowing inert gas through the first purifier 104 strips accumulated solvent from the adsorbent material 105. In some embodiments, the first purifier 104 is heated to a temperature between about 50 C and about 200 C. A heating source 111 may be provided to heat first purifier 104 by either directly heating the first purifier 104, or by heating the inert gas before it enters the first purifier 104. Heating source 111 may be a conventional type heating source, such as a direct contact heater, heat tracing type tape, or another heating source known to one skilled in the art.

When first purifier 104 is regenerated, it may be done selectively such that the flow of acetylene through the distribution system 100 continues and the flow of the purified acetylene stream to the semiconductor processing tool 102 is not interrupted. One method to accomplish this is by diverting the flow from first purifier 104 to second purifier 107 through changing the position of three-way diverter valve 108. Once the flow is diverted, valve 112 may be opened to allow the inert gas from inert gas source 109 and inert gas line 110 to enter the first purifier 104. As mentioned above, the inert gas may be heated by heating source 111, and the flow of inert gas through purifier 104 strips accumulated solvent from the adsorbent material 105. Valve 113 may be opened to allow the inert gas to be sent to vent 116, or collected for further treatment if desired. If necessary, the second purifier 107 may also be regenerated in a similar manner, by diverting flow with diverter valve 108, by sending inert gas to the second purifier 107 through valve 114, and by sending the inert gas to the vent 116 through valve 115. All flows are diverted such that the regenerating of either first purifier 104, or second purifier 107 is done so that the supply of purified acetylene product to semiconductor processing tool 102 is not interrupted.

### EXAMPLES

The following non-limiting examples are provided to further illustrate embodiments of the invention. However, the examples are not intended to be all inclusive and are not intended to limit the scope of the inventions described herein.

### Example 1

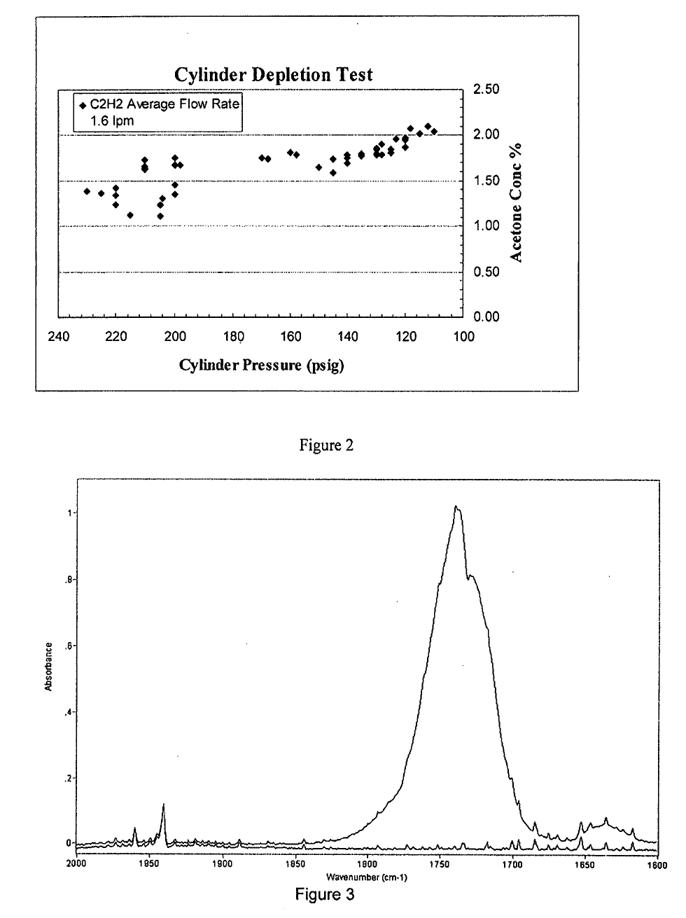

Acetylene from a standard cylinder containing acetylene dissolved in acetone was sent to an FTIR analyzer where the evolution of acetone concentration as a function of pressure was recorded. FIG. 2 shows the results. The acetone concentration was calculated by comparing the FTIR spectra against a known standard of acetone.

### Example 2

Acetylene from a standard cylinder containing acetylene dissolved in acetone was purified according to an embodiment of the current invention.

Process conditions were:
Flow rate—1.5 slpm, pressure-30 psia, residence time—50 sec.

FIG. 3 shows the control FTIR spectra when acetylene stream bypassed the purifier (spectra A). Acetone peak is shown at wave number 1760 cm⁻¹ and comparing with an FTIR spectra of the acetone standard, this peak corresponds to 1.1% (mole) acetone. Spectra B in the figure represent acetylene stream passed through the purifier. The peak in 1760 cm⁻¹ region is similar to the noise level indicating that acetone level was below the detection limit.

While embodiments of this invention have been shown and described, modifications thereof can be made by one skilled in the art without departing from the spirit or teaching of this invention. The embodiments described herein are exemplary only and not limiting. Many variations and modifications of the composition and method are possible and within the scope of the invention. Accordingly the scope of protection is not limited to the embodiments described herein, but is only limited by the claims which follow, the scope of which shall include all equivalents of the subject matter of the claims.

What is claimed is:

1. A method for providing a high purity acetylene product to a semiconductor processing tool, comprising:
a) providing an acetylene source which contains acetylene dissolved in a solvent;
b) withdrawing a stream from the acetylene source, wherein the stream comprises acetylene and the solvent;
c) removing solvent from the withdrawn stream by purifying the withdrawn pressure stream with a first adsorption type purifier to produce a purified acetylene stream; and
d) providing the purified acetylene to at least one semiconductor processing tool.

2. The method of claim 1, further comprising reducing the pressure of the withdrawn stream to less than about 50 psig.

3. The method of claim 1, wherein the withdrawn acetylene stream has a residence time in the first adsorption type purifier between about 30 and 600 seconds.

4. The method of claim 1, further comprising monitoring the purified acetylene stream with a detector.

5. The method of claim 4, further comprising:
a) providing a second adsorption type purifier in parallel to the first adsorption type purifier;
b) switching the flow of the withdrawn pressure stream from the first purifier to the second purifier; and
c) regenerating the first purifier, wherein the first and second purifiers are both adsorbent type purifiers.

6. The method of claim 5, further comprising switching the flow selectively between the first and second purifiers when the detector detects an increase in the amount of solvent present in the purified stream of acetylene.

7. The method of claim 5, further comprising switching the flow selectively between the first and second purifiers based upon the amount of purified acetylene provided to the semiconductor processing tool.

8. The method of claim 5, further comprising regenerating the first purifier by passing an inert gas through the purifier to strip collected solvent from the adsorbent.

9. The method of claim 5, further comprising regenerating the first purifier by heating the purifier bed to a temperature between about 50 C and about 200 C.

10. The method of claim 5, further comprising providing the purified acetylene stream to at least one semiconductor processing tool from the second purifier, while the first purifier is regenerating.

11. The method of claim 1, further comprising purifying the withdrawn pressure stream with a purifier comprising an activated carbon-based adsorbent, wherein the activated carbon-based adsorbent is substantially free of metallic content.

12. The method of claim 11, wherein the activated carbon-based adsorbent comprises less than about 100 ppm of iron.

13. The method of claim 1, further comprising removing particles from the purified acetylene stream with a filter, wherein the purified acetylene stream comprises less than about 1 particle per milliliter which is greater than about 0.3 microns in size.

14. The method of claim 1, wherein the solvent comprises acetone or dimethyl-formamide (DMF).

15. The method of claim 1, further comprising withdrawing the stream from the acetylene storage at a flow rate between about 0.5 lpm and about 20 lpm.

16. The method of claim 1, further comprising removing solvent such that purified acetylene stream comprises less than about 50 ppm of the solvent.

17. The method of claim 2, further comprising reducing the pressure of the withdrawn stream to less than about 20 psig.

18. The method of claim 1, further comprising removing additional impurities from the withdrawn acetylene stream with the first purifier, wherein phosphine and sulfur compounds are each removed such that the purified acetylene stream comprises less than about 1 ppm of each.

19. An apparatus for producing a high purity acetylene product by removing solvent from a stream of acetylene, comprising:
a) an acetylene storage source, wherein the storage source contains acetylene dissolved in a solvent;
b) an acetylene distribution line, which is disposed between the acetylene storage source and a semiconductor processing tool, and which distributes acetylene from the storage source to the semiconductor processing tool;
c) a first acetylene purifier disposed on the distribution line, wherein:
1) the first purifier comprises an adsorption medium suitable to remove at least part of the solvent from the acetylene; and
2) the first purifier receives a stream of acetylene containing solvent from the storage source, and produces a purified stream of acetylene to be sent to the semiconductor processing tool.

20. The apparatus of claim 19, further comprising a pressure reducing device disposed on the distribution line between the acetylene storage source and the first purifier.

21. The apparatus of claim 19, further comprising: a detector, disposed on the distribution line after the first purifier, wherein the detector is suitable to monitor the purified stream of acetylene.

22. The apparatus of claim 19, further comprising a second purifier, wherein
a) the second purifier is disposed on the distribution line in parallel to the first purifier; and
b) the first and second purifiers both comprise an activated carbon-based adsorbent which is substantially free of metallic content.

23. The apparatus of claim 22, further comprising:
a) a diverter valve, suitable to divert the flow of acetylene from the storage source to either the first or second purifier; and
b) a source of inert gas and an inert gas line which connects the source of inert gas to the first and second purifiers so as to allow for inert gas to selectively flow through a purifier when the flow of acetylene is diverted from that purifier.

24. The apparatus of claim 19, further comprising a flow controller disposed on the distribution line after the first purifier and before the semiconductor processing tool.

25. The apparatus of claim 23, further comprising a heating source, wherein the heating source is suitable to heat the inert gas, the first purifier, or the second purifier to a temperature between about 50 C. and about 200 C.

26. The apparatus of claim 19, wherein the purified stream of acetylene contains less than about 50 ppm of the solvent.

27. The apparatus of claim 19, wherein the solvent comprises acetone or dimethyl-formamide (DMF).

28. The apparatus of claim 28, wherein the pressure reducing device reduces the pressure of the withdrawn stream of acetylene containing solvent to less than about 50 psig.

29. The apparatus of claim 28, wherein the pressure reducing device reduces the pressure of the withdrawn stream of acetylene containing solvent to less than about 20 psig.

30. The apparatus of claim 19, further comprising a particle filter located downstream of the first purifier.
